(19) United States
(12) Patent Application Publication
(10) Pub. No.: US 2006/0064148 A1
(43) Pub. Date: Mar. 23, 2006

(54) Self-adherent instant cold compress therapeutic wrap

(76) Inventors: Domenic A. Sammarco, New Canaan, CT (US); Louis A. Visconti, Stamford, CT (US)

Correspondence Address: Domenic A. Sammarco, 54 Toquam Road, New Canaan, CT, 06840, US

(21) Appl. No.: 10/991,642

(22) Filed: Nov. 19, 2004

Related U.S. Application Data
(60) Provisional application No. 60/612,117, filed on Sep. 23, 2004.

(51) Int. Cl. A61F 7/00

(52) U.S. Cl. 607/112; 607/114

(57) ABSTRACT

A compact and disposable cold compress wrap which combines the use of cold therapy (a 6¼″ wide and 8½″ high) instant ice pack consisting of ammonium nitrate and water creating an endothermic reaction which shall generate a cryotherapeutic (cold therapy) environment. Along with a self adherent 4″ wide blue elastic bandage which shall create compression. In combination both components shall produce a compression cryotherapeutic product.

### CROSS REFERENCE TO RELATED APPLICATIONS

This application claims the benefit of provisional patent application Ser. No. 60/612,117 filed 2004 Sep. 23 by the present inventors.

### FEDERALLY SPONSORED RESEARCH

Not Applicable

### SEQUENCE LISTING OR PROGRAM

Not Applicable

### BACKGROUND OF THE INVENTION

1. Field of Invention

The invention relates to cryogenic cold compress therapeutic wraps for sports injuries.

2. Prior Art

The invention was developed to satisfy a need for treating sports injuries effectively and quickly. The self-adherent cold compress therapeutic wrap is designed to be used anywhere on an injured body requiring relief by cold therapy and compression. The invention is a combination of two prior arts which are Application 495289 Instant Reusable Compress Jun. 28, 1995 and Application 940221 Elastic Bandage Self-Adherent Aug. 27, 2001. It is an improvement over the prior art. Chemical cold bags contain separate components which mix in an endothermic reaction resulting in 5 to 15 minutes of cold therapy. External compression of an injured area helps limit and reduce bleeding and edema and controls initial swelling by reducing the blood supply and interstitial fluids to the injured area. PRICE Treatment for Acute Soft Tissue Injury:
P=Protect the area from further injury using a splint, sling, crutches, etc.
R=Rest the injured part to promote tissue healing
I=Ice (i.e., apply cryotherapy to) the injured part
C=Compress the injured area (e.g. with an elastic bandage)
E=Elevate the injured part above the level of the heart

Ice. By decreasing tissue temperature, the application of cold (usually through the application of ice) is thought to diminish pain, local tissue metabolism, and muscle spasm and minimize the inflammatory response, thereby aiding recovery. Cryotherapy should begin as soon as possible after injury occurs, preferably within 10 to 15 minutes. The safest effective duration of application is 20 to 30 minutes.

Compression. External compression of the injured area helps to limit and reduce bleeding and edema and control initial swelling. The application of gentle but firm pressure around the injured area decreases the amount of space available for swelling and pushes interstitial fluid back into the capillaries and lymph vessels. It also reduces the blood supply by about 95% within a few seconds.

The most common means of compression is the elastic bandage or wrap (often referred to as an “Ace bandage”.) Compression should be applied for at least 72 hours after an injury, both during and after cold application.

### OBJECTS AND ADVANTAGES

Several objects and advantage of the invention are:
a.) to provide a sanitary and efficient means to treat sports injuries.
b.) to provide firm control of swelling resulting from sport injury trauma.
c.) to provide a means to minimize pain from injury with cold therapy.
d.) to provide a means of protecting area from further injury.

### SUMMARY

The invention is called a CCT Wrap (Cold Compression Therapeutic Wrap) and it combines the two most important aspects of cold therapy—temperature and compression in one complete product. It is sanitary and disposable.

### DRAWINGS—FIGURE

FIG. 1 is a top view of the CCT Wrap showing ice pack with self adherent bandage attached to it.

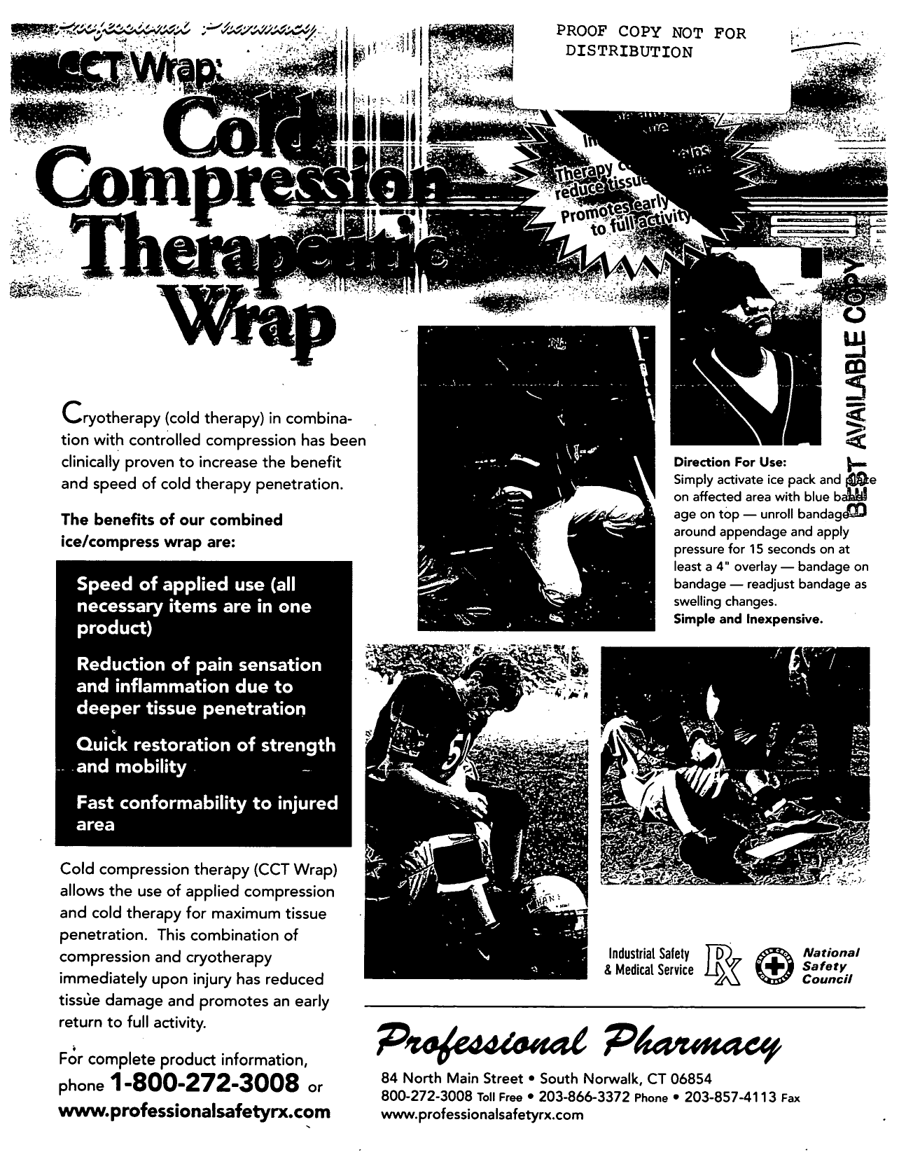

### DETAILED DESCRIPTION

The ice pack¹¹ is an instant cold compress disposable bag commonly called an instant ice pack containing ammonium nitrate and water in two sealed containers. It is approximately 6¼″ wide and 8½″ high. A 24″ section of self adherent 4″ wide blue elastic bandage¹² is adhered to one side of instant ice pack ready for use. The bandage tail ends are pre-rolled on each end to be prepared for immediate use.

### REFERENCE NUMBERS

11. Ice pack
12. Self-adherent bandage
   Operation

Use of the CCT Wrap is very easy. The ice pack is applied to the injury area with the self-adherent bandage unrolled and its ends fastened via the self stick properties impregnated into the bandage material around the injury area.

I claim:

1. A 24″ section of blue self-adherent bandage attached to an ice pack with an adhesive
Said ends of self-adherent bandage are used to compress the ice pack component onto the injury area
Whereby CCT Wrap will help soothe the injury area as short a time as possible so as to return injured individual to full activity.
